(19) United States
(12) Patent Application Publication
(10) Pub. No.: US 2015/0364340 A1
(43) Pub. Date: Dec. 17, 2015

(54) CHEMICAL SENSOR ARRAYS FOR ODOR DETECTION

(71) Applicant: EMPIRE TECHNOLOGY DEVELOPMENT LLC, Wilmington, DE (US)

(72) Inventor: Masahiro UEDA, Kyoto (JP)

(21) Appl. No.: 14/765,561

(22) PCT Filed: Feb. 5, 2013
(86) PCT No.: PCT/US2013/024778

§ 371 Date: Aug. 3, 2015

(51) Int. Cl. H01L 21/388; G01N 33/00; G01N 27/414; H01L 21/477; H01L 21/02

(52) U.S. Cl. CPC H01L 21/388; H01L 21/477; H01L 21/02565; H01L 21/02603; H01L 21/02625; H01L 21/02664; G01N 27/4145; G01N 33/0001

(57) ABSTRACT

An array of semiconductor chemical sensors and a method for manufacturing the array of semiconductor chemical sensors are disclosed. In some examples, the method may include providing a semiconductor substrate including a plurality of areas, and ejecting onto each area of the semiconductor substrate a solution including at least one modification material for modifying each area of the semiconductor substrate.

### BACKGROUND

Unless otherwise indicated herein, the materials described herein are not prior art to the claims in the present application and are not admitted to be prior art by inclusion in this section.

Odor is produced by volatile organic compounds. A variety of sensors, including a chemical sensor, a biosensor, a mass spectrometer, a differential optical absorption spectrometer, etc., are available for detecting and identifying odor. A chemical sensor, among others, detects odor molecules based on chemical reaction between the odor molecules and sensing materials disposed on a surface of the sensor. Such chemical reaction triggers a certain change in physical properties of the sensing materials, which is converted to an electrical signal.

### SUMMARY

Some embodiments disclosed herein include a method for manufacturing an array of semiconductor chemical sensors. In some embodiments, the method may include providing a semiconductor substrate including a plurality of areas; and ejecting onto each area of the semiconductor substrate a solution including at least one modification material for modifying each area of the semiconductor substrate. In some embodiments, the ejecting may be performed by a nozzle of an inkjet printer.

In some embodiments, the modification material may include a compound that has a selective affinity for a chemical to be detected. By way of example, but not limitation, the modification material may include at least one of Nafion, polyethyleneimine, polyaniline, polypyrrole, polythiophene, sodium polystyrene sulfonate, and palladium.

In some embodiments, the method may further include determining an amount of the solution to be ejected onto each area of the semiconductor substrate. The determined amount of the solution may be ejected onto each area of the semiconductor substrate.

In some embodiments, the semiconductor substrate may be provided by sintering microparticles of an oxide semiconductor material. By way of example, but not limitation, the oxide semiconductor material may include at least one of SnO₂, TiO₂, and ZnO.

In some embodiments, the semiconductor substrate may be provided by fabricating nanofibers of an oxide semiconductor material by electrospinning. By way of example, but not limitation, the oxide semiconductor material may include TiO₂.

In some embodiments, the semiconductor substrate may be provided by anodizing an oxide semiconductor material. By way of example, but not limitation, the oxide semiconductor material may include TiO₂; and the solution may include at least one solvent selected from the group consisting of water, ethyleneglycol, and an amino alcohol. In some embodiments, the solution in which the modification material having a residue of a silane coupling agent is dispersed in a polar organic solvent may be ejected onto each area of the semiconductor substrate.

In some embodiments, the semiconductor substrate may be provided by forming a layer of carbon nanotubes. By way of example, but not limitation, the solution may include at least one solvent selected from the group consisting of dimethylformamide (DMF), N-methylpyrrolidone (NMP), water, and water with a surfactant; and the surfactant may include at least one of sodium benzenesulfonate (NaBS), gum arabic, and cyclodextrin. In some embodiments, the solution in which the modification material with a pendant pyrene residue is dispersed in a polar organic solvent may be ejected onto each area of the semiconductor substrate. In some embodiments, the solution including a diazonium compound of the modification material may be ejected onto each area of the semiconductor substrate. In some embodiments, the solution including a nitrene compound of the modification material may be ejected onto each area of the semiconductor substrate. In some embodiments, the solution including an azomethine ylide compound of the modification material may be ejected onto each area of the semiconductor substrate. In some embodiments, the solution including a carbene compound of the modification material may be ejected onto each area of the semiconductor substrate.

Also provided is an array of semiconductor chemical sensors manufactured by any of the methods provided herein.

Also provided is an odor sensor including an array of semiconductor chemical sensors manufactured by any of the methods provided herein.

Alternative embodiments disclosed herein may include an array of semiconductor chemical sensors. In some embodiments, the array may include a semiconductor substrate including a plurality of areas, each area of the semiconductor substrate being associated with each element of the array of semiconductor chemical sensors; and at least one modification material printed on the semiconductor substrate. In some embodiments, an amount of the modification material printed on the semiconductor substrate may vary according to the area of the semiconductor substrate.

Yet alternative embodiments disclosed herein may include an apparatus for manufacturing an array of semiconductor chemical sensors. In some embodiments, the apparatus may include a substrate holder configured to hold a semiconductor substrate, a nozzle configured to eject onto each area of the semiconductor substrate a solution including at least one modification material for modifying each area of the semiconductor substrate held by the substrate holder, and a controller configured to control at least one of an ejection pressure and an ejection amount of the nozzle. In some embodiments, the controller may be further configured to control drying of the semiconductor substrate onto which the solution including the modification material has been applied.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIGS. 1A-1C schematically show an illustrative example of a process of manufacturing an array of semiconductor chemical sensors, arranged in accordance with at least some embodiments described herein;

FIG. 2 schematically shows an illustrative example of a circuit for implementing each sensor element of an array of semiconductor chemical sensors, arranged in accordance with at least some embodiments described herein;

FIG. 3 schematically shows an illustrative example of a process of manufacturing an array of semiconductor chemical sensors, arranged in accordance with at least some embodiments described herein;

FIG. 4 schematically shows another illustrative example of a process of manufacturing an array of semiconductor chemical sensors, arranged in accordance with at least some embodiments described herein;

FIGS. 5A-5C schematically show illustrative examples of structures in each of which a modification material is covalently bonded to a semiconductor substrate, arranged in accordance with at least some embodiments described herein; and

FIGS. 6A-6D schematically show illustrative examples of odor detection patterns, arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the drawings, can be arranged, substituted, combined, separated, and designed in a wide variety of different configurations, all of which are explicitly contemplated herein.

Technologies are herein generally described an array of semiconductor chemical sensors for odor detection.

In some examples, the array of semiconductor chemical sensors may be fabricated by ejecting onto a semiconductor substrate a solution including at least one modification material for modifying each area of the semiconductor substrate. Each area of the semiconductor substrate onto which the at least one modification material is ejected may form each sensor element of the array of semiconductor chemical sensors. The at least one modification material may include any compound that has a selective affinity for a chemical or gas to be detected.

In some examples, the solution including the at least one modification material may be ejected onto each area of the semiconductor substrate by a nozzle of an inkjet printer. Using the inkjet printer with high resolution, it may be possible to provide different types of chemical modification to each small sensor element of the array. By way of example, but not limitation, when using a high-precision inkjet printer having a resolution up to 9600×2400 dpi or 5760×1440 dpi (corresponding to about 3-17 μm), a semiconductor substrate of size of 1 cm×1 cm may be made to a sensor array including 40,000 chemical sensor elements by dividing the semiconductor substrate into 40,000 elements (that is, 200×200 elements, each of which has size of 50 μm×50 μm), and providing 40,000 types of chemical modification onto each element. Such sensor array may detect and/or identify a gas (even a gas at very low concentration or complex mixed gases) through pattern recognition.

### Fabrication of Chemical Sensor Arrays

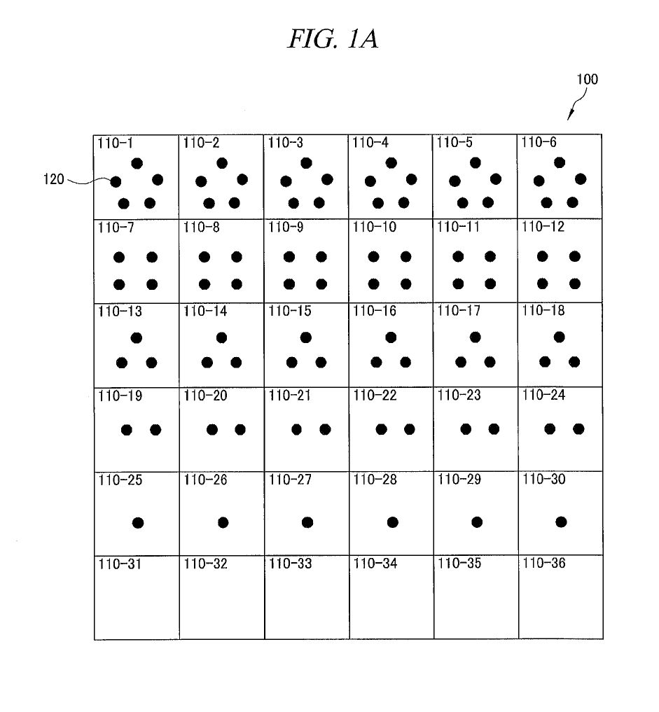

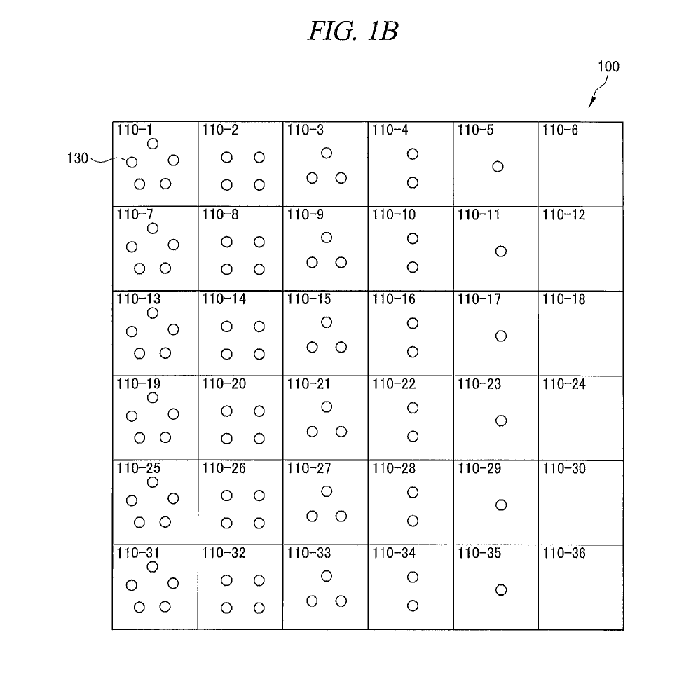

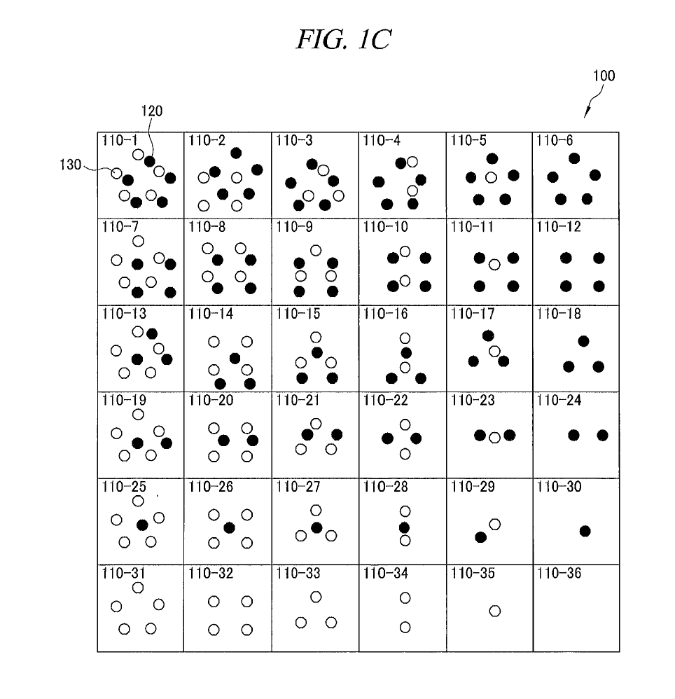

In some embodiments, an array of semiconductor chemical sensors may be fabricated by providing at least one modification material onto a semiconductor substrate. FIGS. 1A-1C schematically show an illustrative example of a process of manufacturing an array of semiconductor chemical sensors, arranged in accordance with at least some embodiments described herein.

As depicted in FIGS. 1A-1C, a semiconductor substrate 100 may include multiple areas 110-1, 110-2, . . . , 110-36. In some embodiments, semiconductor substrate 100 may be made to a sensor array 100 including multiple sensor elements 110-1, 110-2, . . . , 110-36 (collectively, sensor element 110), by providing a first modification material 120 (as in FIG. 1A) and a second modification material 130 (as in FIG. 1B) onto each of areas 110-1, 110-2, . . . , 110-36. Each of first modification material 120 and second modification material 130 may have a selective affinity for at least one chemical to be detected.

In some embodiments, the providing of first modification material 120 and second modification material 130 onto areas 110-1, 110-2, . . . , 110-36 may respectively include ejecting a first solution including first modification material 120 and a second solution including second modification material 130 onto areas 110-1, 110-2, . . . , 110-36, for example, by a nozzle of an inkjet printer (not shown). In such cases, an ejection pressure and/or an ejection amount of the nozzle may be adjusted depending on the desired implementation, for example, by a controller (not shown) which may be operatively coupled to the nozzle.

As depicted in FIG. 1A, first modification material 120 may be provided onto semiconductor substrate 100. The amount of first modification material 120 may be different for each of areas 110-1, 110-2, . . . , 110-36. By way of example, but not limitation, the amount of first modification material 120 may gradually increase from bottom to top of semiconductor substrate 100, as in FIG. 1A.

Then, as depicted in FIG. 1B, second modification material 130 may be provided onto semiconductor substrate 100. The amount of second modification material 130 may be different for each of areas 110-1, 110-2, . . . , 110-36. By way of example, but not limitation, the amount of second modification material 130 may gradually increase from right to left of semiconductor substrate 100, as in FIG. 1B.

The providing of first modification material 120 as in FIG. 1A and the providing of second modification material 130 as in FIG. 1B may result in sensor array 100 as depicted in FIG. 1C. Sensor array 100 may have thirty-six (36) different combinations of first modification material 120 and second modification material 130 to detect ambient chemicals and/or odors. That is, sensor array 100 may have 36 different sensor elements 110-1, 110-2, . . . , 110-36.

Although FIGS. 1A-1C illustrates that sensor array 100 includes 36 (that is, 6×6) sensor elements, those skilled in the art will recognize that sensor array 100 may include any number of sensor elements. Also, although FIGS. 1A-1C illustrates that two modification materials are employed to fabricate sensor array 100, those skilled in the art will recognize that any number of modification materials may be employed to fabricate sensor array 100.

### Apparatus for Manufacturing Chemical Sensor Arrays

In some embodiments, an apparatus for manufacturing an array of chemical sensors may include a substrate holder configured to hold a semiconductor substrate, and a nozzle configured to eject onto each area of the semiconductor substrate a solution including at least one modification material for modifying each area of the semiconductor substrate. In some embodiments, the apparatus may further include a controller configured to control or adjust operating parameters of the nozzle, including at least one of an ejection pressure and an ejection amount of the nozzle. In some embodiments, the controller may also be configured to control drying condition of the semiconductor substrate after the solution including the modification material has been applied onto the semiconductor substrate by the nozzle.

### Sensor Circuits

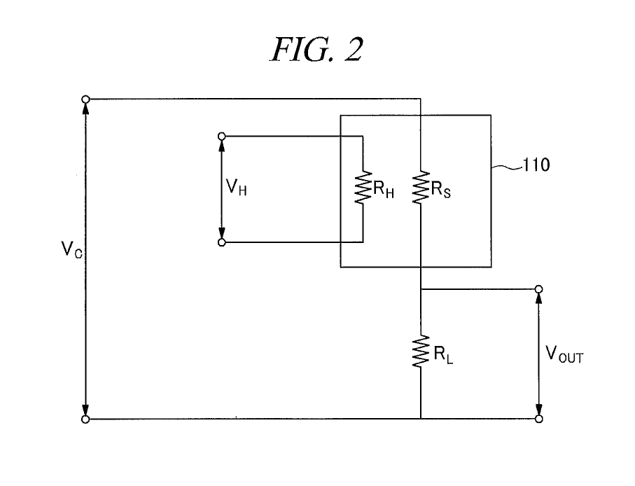

In some embodiments, each of sensor elements of a sensor array may be implemented by an electric circuit. FIG. 2 schematically shows an illustrative example of a circuit for implementing each sensor element of an array of semiconductor chemical sensors, arranged in accordance with at least some embodiments described herein.

As depicted, a predetermined circuit voltage V_{C} may be applied to sensor element 110 and a load resistance R_{L} connected in series with sensor element 110. Further, a predetermined heater voltage V_{H} may be applied to a heater resistance R_{H} to heat sensor element 110 to a desired temperature to detect a target chemical.

In some embodiments, by measuring an output voltage V_{OUT}, a sensor resistance Rₛ of sensor element 110 may be calculated as follows: R_{S}=((V_{C}−V_{OUT})/V_{OUT})×R_{L}. In such cases, a concentration of the target chemical detected by sensor element 110 may be determined based on the calculated value of sensor resistance Rₛ, since sensor resistance Rₛ of sensor element 110 may vary depending on a concentration of the target chemical detected by sensor element 110.

### Preparation of Semiconductor Substrates

As the size of a sensor element decreases for more dense integration, the surface area of the sensor element for detecting ambient chemicals decreases, and thus the sensitivity for the ambient chemicals also decreases. In this regard, in some embodiments, the semiconductor substrate may include a sintered product of an oxide semiconductor material, nanofibers or nanorods of an oxide semiconductor material, an anodized product of an oxide semiconductor material, and/or carbon nanotubes (CNTs), to enhance the sensitivity of the sensor element.

In some embodiments, the semiconductor substrate may be fabricated by sintering microparticles of an oxide semiconductor material. By sintering the microparticles of the oxide semiconductor material, specific surface area of the semiconductor substrate may increase. By way of example, but not limitation, the oxide semiconductor material may include SnO₂ (tin dioxide), TiO₂ (titanium dioxide), ZnO (zinc oxide), or combination thereof, etc. By way of example, but not limitation, the size of the microparticles may be tens of nanometers.

In some embodiments, the semiconductor substrate may be fabricated by nanofibers or nanorods of an oxide semiconductor material (e.g., TiO₂, etc.). In some embodiments, the nanofibers or nanorods of oxide semiconductor material may be formed by an electrospinning process. By way of example, but not limitation, polyaniline may be further adsorbed on the surface of TiO₂ nanofibers or nanorods. By way of example, but not limitation, the diameters of the nanofibers or nanorods may be in the range between tens of nanometers and about 200 nm. Specific examples of diameters include about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, and ranges between any two of these values (including endpoints).

In some embodiments, the semiconductor substrate may be fabricated by anodizing at least one oxide semiconductor material (e.g., TiO₂, etc.). By anodizing the oxide semiconductor material, specific surface area of the semiconductor substrate may increase. Further, by anodizing the oxide semiconductor material, it may be possible to control pore properties of the semiconductor substrate, such as pore diameter, pore gap and/or pore depth, in a simple manner.

In some embodiments, the semiconductor substrate may be fabricated by forming a layer of carbon nanotubes (CNTs). The carbon nanotube itself may act as a chemical sensor. By way of example, but not limitation, the layer of carbon nanotubes may be formed by placing a number of carbon nanotubes between two electrodes. By way of another example, but not limitation, the layer of carbon nanotubes may be formed by placing electrodes on a buckypaper of carbon nanotubes.

Preparation of Solutions Including Modification Materials to be Provided onto Semiconductor Substrates

A solution to be ejected onto a semiconductor substrate may include at least one modification material, and at least one solvent which may dissolve the at least one modification material and adhere well to the semiconductor substrate.

In some embodiments, the modification material may modify each area of the semiconductor substrate to have a selective affinity for at least one chemical to be detected. By way of example, but not limitation, the modification material may include a polymer (e.g., Nafion, polyethyleneimine, polyaniline, polypyrrole, polythiophene, sodium polystyrene sulfonate, etc.), and/or a metal (e.g., palladium, etc.). By applying polymers or metallic microparticles onto a carbon nanotube substrate, selectivity and/or sensitivity for the chemical to be detected may be improved.

In some embodiments, the solvent may be determined based at least in part on surface tension, viscosity, and/or polarity. By way of example, but not limitation, the solvent may be water, or a hydrophilic organic solvent that has hydrogen bond properties or that may form a metal coordination structure (e.g., ethyleneglycol, an amino alcohol, etc.) for an anodized oxide semiconductor substrate. By way of example, but not limitation, the solvent may be dimethylformamide (DMF), N-methylpyrrolidone (NMP), chloroform (CHCl₃), o-dichlorobenzene (o-DCB), water, or combinations thereof, for the carbon nanotube substrate. When using water as the solvent, a surfactant (e.g., sodium benzenesulfonate (NaBS), gum arabic, cyclodextrin, etc.) may be added to the solution.

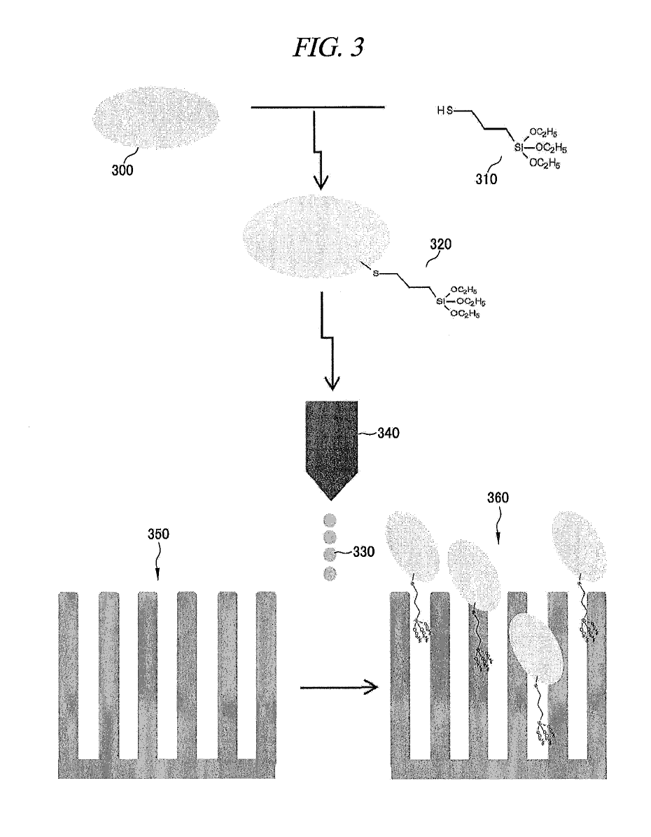

In some embodiments, a silane coupling agent may be used for adsorbing the modification material to the anodized oxide semiconductor substrate, as illustrated in FIG. 3. Referring to FIG. 3, a modification material 300 may be bonded with a silane coupling agent 310, thereby providing a composite 320 of modification material 300 having a residue of silane coupling agent 310. Then, a solution 330 in which composite 320 is dispersed in a polar organic solvent or water may be ejected onto an anodized oxide semiconductor substrate 350 by a nozzle 340. This may provide a sensor element 360, in which modification material 300 may be adsorbed to the surface of anodized oxide semiconductor substrate 350.

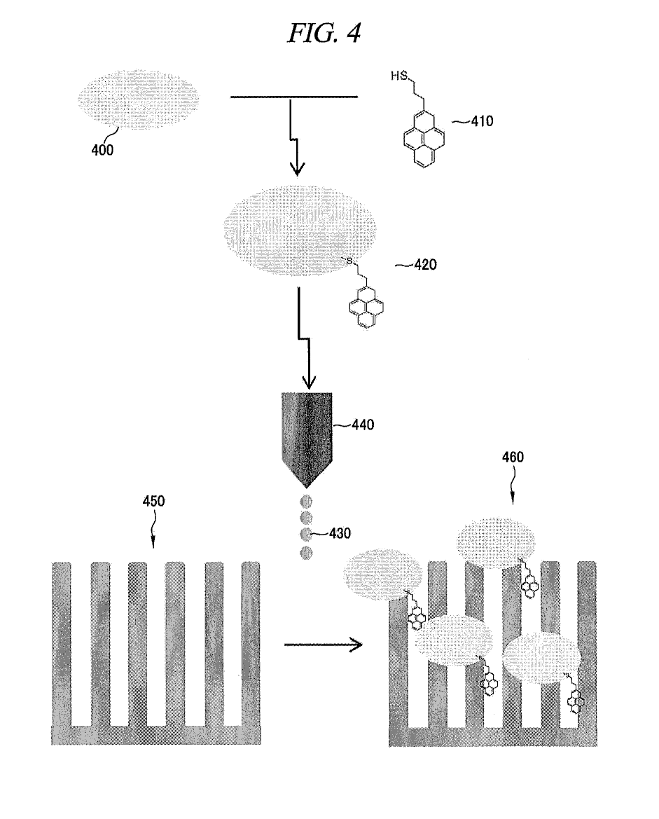

In some embodiments, pyrene may be used for adsorbing the modification material to the carbon nanotube substrate, as illustrated in FIG. 4. Referring to FIG. 4, a modification material 400 may be bonded with a pyrene derivative 410, thereby providing a composite 420 of modification material 400 having a pendant pyrene residue. Then, a solution 430 in which composite 420 is dispersed in a polar organic solvent (e.g., dimethylformamide (DMF), etc.) may be ejected onto a carbon nanotube substrate 450 by a nozzle 440. This may provide a sensor element 460, in which modification material 400 may be adsorbed to the surface of carbon nanotube substrate 450.

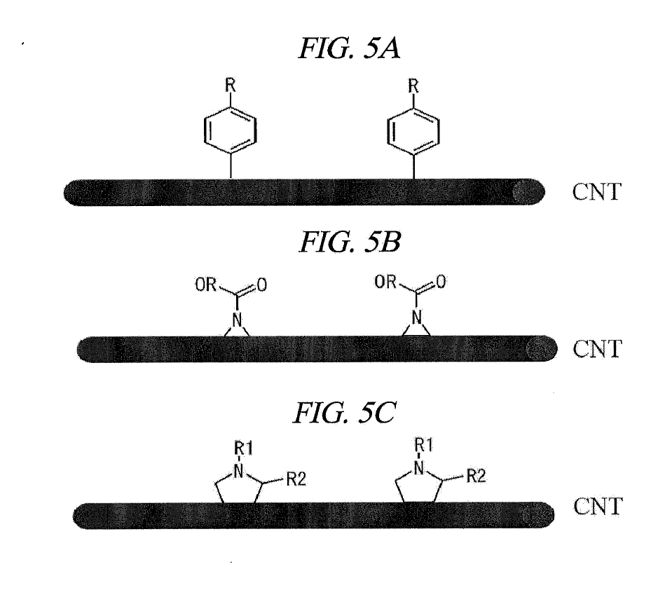

In some embodiments, the modification material may be covalently bonded to the carbon nanotube substrate, as illustrated in FIGS. 5A-5C. By way of example, but not limitation, a diazonium compound of the modification material (as in FIG. 5A), a nitrene compound of the modification material (as in FIG. 5B), an azomethine ylide compound of the modification material (as in FIG. 5C), and/or a carbene compound of the modification material may be bonded to the carbon nanotube substrate. In such cases, a reaction time may be required after ejection of the compound of the modification material. In FIGS. 5A-5C, R, R1 and R2 may respectively denote a desired modification material.

### EXAMPLES

The present disclosure will be understood more readily by reference to the following examples, which are provided by way of illustration and are not intended to be limiting in any way.

### Example 1

### Preparation of Semiconductor Substrates

A sintered SnO₂ substrate is prepared by sintering microparticles of SnO₂. A TiO₂ nanofiber substrate is prepared by electrospinning and sintering at a temperature of 600° C. An anodized TiO₂ substrate is prepared by two phases of oxidation in the presence of negative fluorine ions in ethylene glycol. A carbon nanotube (CNT) substrate is a single layer of carbon nanotubes in a form of buckypaper prepared by an arc discharge method.

### Example 2

### Determination of Solutions to be Ejected onto Semiconductor Substrates, Operating Parameters of a Nozzle, and Drying Conditions

With taking into consideration of types and/or materials of the semiconductor substrates, a solution to be ejected onto each of the semiconductor substrates (a solute (that is, a modification material), a solvent, solid content, and an additive (if any)) is determined as in the table below.

| | Semiconductor Substrate | | | | | | | |
|---|---|---|---|---|---|---|---|---|
| | | | | Specific | Solution | | | |
| | | | Particle | Surface | | | Solid | |
| | | | Diameter | Area | | | Content | |
| No. | Type | Material | (nm) | (m²/g) | Solute | Solvent | (ppm) | Additive |
| (1) | Sintered | SnO₂ | about 60 | about 500 | Nafion | water/n-propyl | 100 | — |
| | | | | | | alcohol | | |
| (2) | Sintered | SnO₂ | about 60 | about 500 | polyethyleneimine | water | 100 | — |
| (3) | Nano- | TiO₂ | about | about 300 | polyaniline | dimethylformamide | 50 | — |
| | fiber | | 200 | | | (DMF) | | |
| (4) | Anodized | TiO₂ | pore | about 700 | sodium | water | 200 | — |
| | | | diameter: | | polystyrene | | | |
| | | | about 30; | | sulfonate | | | |
| | | | pore gap: | | (NaPSS) | | | |
| | | | about 20; | | | | | |
| | | | pore depth: | | | | | |
| | | | about 100 | | | | | |
| (5) | CNT | C | about 1 | about 600 | polyethyleneimine | dimethylformamide | 100 | — |
| | | | | | | (DMF) | | |
| (6) | CNT | C | about 1 | about 600 | polypyrrole | dimethylformamide | 100 | — |
| | | | | | | (DMF) | | |
| (7) | CNT | C | about 1 | about 600 | polypyrrole | N- | 100 | — |
| | | | | | | methylpyrrolidone | | |
| | | | | | | (NMP) | | |
| (8) | CNT | C | about 1 | about 600 | sodium | water | 100 | sodium |
| | | | | | polystyrene | | | benzenesulfonate |
| | | | | | sulfonate | | | (NaBS) |
| | | | | | (NaPSS) | | | |
| (9) | CNT | C | about 1 | about 600 | sodium | water | 100 | gum arabic |
| | | | | | polystyrene | | | |
| | | | | | sulfonate | | | |
| | | | | | (NaPSS) | | | |

Operating parameters (an ejection pressure and an ejection amount) of a nozzle, and drying conditions (temperature and time duration) are also determined for the above (1)-(9). For (1)-(4), the ejection pressure of the nozzle is determined as 0.8 kPa, and the ejection amount is determined as 6.5 pL; while for (5)-(9), the ejection pressure of the nozzle is determined as 1.2 kPa, and the ejection amount is determined as 7.5 pL. For (1)-(4), the drying temperature is determined as 40° C., and the drying time duration is determined as 3 minutes; while for (5)-(9), the drying temperature is determined as 60° C., and the drying time duration is determined as 1 minute.

### Example 3

### Pattern Recognition for Odor Sensing

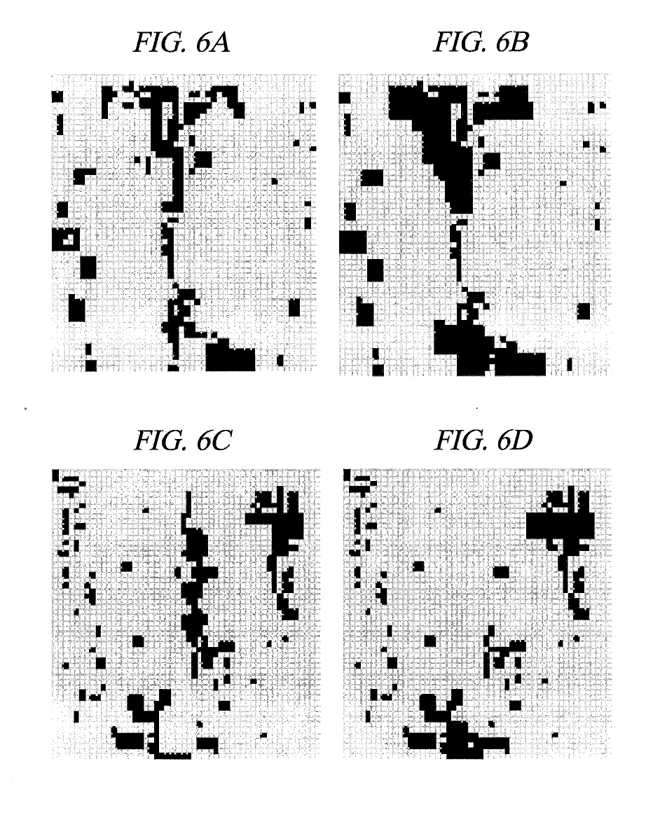

A sensor array including 50×50 sensor elements detects ambient chemical(s) and provides odor detection patterns as shown in FIGS. 6A-6D. In FIGS. 6A-6D, black dots represent the sensor elements that detect a corresponding target chemical of a concentration not less than 50 ppm.

FIG. 6A is an odor detection pattern of a wine from a first winery, while FIG. 6B is an odor detection pattern of a wine from a second winery. In such cases, the wines from multiple wineries may be distinguished from each other by comparing the odor detection patterns. Similarly, FIG. 6C is an odor detection pattern of an eel produced in country A, while FIG. 6D is an odor detection pattern of an eel produced in country B. In such cases, falsification of origin may be proved by comparing the odor detection patterns.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one or one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for manufacturing an array of semiconductor chemical sensors, the method comprising:
providing a semiconductor substrate including a plurality of areas; and
ejecting onto each area of the semiconductor substrate a solution including at least one modification material for modifying each area of the semiconductor substrate,
wherein the modification material comprises a compound that has a selective affinity for a chemical to be detected.

2. The method of claim 1, wherein the modification material further comprises a second compound that has a selective affinity for a second chemical to be detected.

3. The method of claim 1, wherein the modification material comprises at least one of Nafion, polyethyleneimine, polyaniline, polypyrrole, polythiophene, sodium polystyrene sulfonate, and palladium.

4. The method of claim 1, further comprising:
determining an amount of the solution to be ejected onto each area of the semiconductor substrate,
wherein the ejecting comprises ejecting onto each area of the semiconductor substrate the determined amount of the solution,
wherein the determined amount of the solution varies between each area of the plurality of areas.

5. The method of claim 1, wherein the providing the semiconductor substrate comprises sintering microparticles of an oxide semiconductor material.

6. The method of claim 5, wherein the oxide semiconductor material comprises at least one of SnO₂, TiO₂, and ZnO.

7. The method of claim 1, wherein the providing the semiconductor substrate comprises fabricating nanofibers of an oxide semiconductor material by electrospinning.

8. The method of claim 7, wherein the oxide semiconductor material comprises TiO₂.

9. The method of claim 1, wherein the providing the semiconductor substrate comprises anodizing an oxide semiconductor material.

10. The method of claim 9, wherein the oxide semiconductor material comprises TiO₂.

11. The method of claim 9, wherein the solution comprises at least one solvent selected from the group consisting of water, ethyleneglycol, and an amino alcohol.

12. The method of claim 9, wherein the ejecting comprises ejecting onto each area of the semiconductor substrate the solution in which the modification material having a residue of a silane coupling agent is dispersed in a polar organic solvent.

13. The method of claim 1, wherein the providing the semiconductor substrate comprises forming a layer of carbon nanotubes.

14. The method of claim 13, wherein the solution comprises at least one solvent selected from the group consisting of dimethylformamide (DMF), N-methylpyrrolidone (NMP), water, and water with a surfactant.

15. The method of claim 14, wherein the surfactant comprises at least one of sodium benzenesulfonate (NaBS), gum arabic, and cyclodextrin.

16. The method of claim 13, wherein the ejecting comprises ejecting onto each area of the semiconductor substrate the solution in which the modification material with a pendant pyrene residue is dispersed in a polar organic solvent.

17. The method of claim 13, wherein the ejecting comprises ejecting onto each area of the semiconductor substrate the solution including a diazonium compound of the modification material.

18. The method of claim 13, wherein the ejecting comprises ejecting onto each area of the semiconductor substrate the solution including a nitrene compound of the modification material.

19. The method of claim 13, wherein the ejecting comprises ejecting onto each area of the semiconductor substrate the solution including an azomethine ylide compound of the modification material.

20. The method of claim 13, wherein the ejecting comprises ejecting onto each area of the semiconductor substrate the solution including a carbene compound of the modification material.

21. The method of claim 1, wherein the ejecting is performed by a nozzle of an inkjet printer.

22. An array of semiconductor chemical sensors manufactured by the method of claim 1.

23. An odor sensor comprising the array of semiconductor chemical sensors of claim 22.

24. An array of semiconductor chemical sensors, the array comprising:
a semiconductor substrate including a plurality of areas, each area of the semiconductor substrate being associated with each element of the array of semiconductor chemical sensors; and
at least one modification material printed on the semiconductor substrate,
wherein an amount of the modification material printed on the semiconductor substrate varies according to a position of the area on the semiconductor substrate.

25. An apparatus comprising:
a substrate holder configured to hold a semiconductor substrate;
a nozzle configured to eject onto each area of the semiconductor substrate a solution including at least one modification material for modifying each area of the semiconductor substrate held by the substrate holder; and
a controller configured to control at least one of an ejection pressure and an ejection amount of the nozzle,
wherein the apparatus is configured to manufacture an array of semiconductor chemical sensors.

26. The apparatus of claim 25, wherein the controller is further configured to control drying of the semiconductor substrate onto which the solution including the modification material has been applied.
